(19) United States
(12) Patent Application Publication
(10) Pub. No.: US 2023/0389837 A1
(43) Pub. Date: Dec. 7, 2023

(54) ANALYTE SENSOR FOR MEASURING AT VARYING DEPTHS WITHIN A USER

(71) Applicant: Cercacor Laboratories, Inc., Irvine, CA (US)

(72) Inventors: Venkatramanan Krishnamani, Irvine, CA (US); Anando Devadoss, Irvine, CA (US); Sahand Pirbadian, Irvine, CA (US); Nan Hu, Irvine, CA (US)

(21) Appl. No.: 18/143,469

(22) Filed: May 4, 2023

Related U.S. Application Data
(60) Provisional application No. 63/364,243, filed on May 5, 2022.
(60) Provisional application No. 63/480,739, filed on Jan. 20, 2023.

(51) Int. Cl. A61B 5/1486; A61B 5/145

(52) U.S. Cl. CPC A61B 5/14865; A61B 5/14532

(57) ABSTRACT

A physiological sensor system that may include an analyte sensor configured to measure at least one analyte at varying depths at a tissue site of a user. The analyte sensor may include a plurality of electrodes patterned on a semi-rigid substrate meant to penetrate the skin of a user. The substrate may be varying lengths to increase the number of electrodes at the penetration site of the user and decrease the likelihood of a bad reading due to poor placement of the electrodes. The electrodes may be patterned on both sides of the substrate to increase number of measurement sites. The analyte sensor can be positioned in an electrochemical cell disposed in the body of the user.

### CROSS-REFERENCE TO RELATED APPLICATIONS

This application claims benefit of priority under 35 U.S.C. § 119(e) to U.S. Provisional Application No. 63/364,243, filed on May 5, 2022, entitled “ANALYTE SENSOR,” which is hereby incorporated by reference in its entirety, and to U.S. Provisional Application No. 63/480,739, filed on Jan. 20, 2023, entitled “AN ANALYTE SENSOR FOR MEASURING AT VARYING DEPTHS WITHIN A PATIENT,” which is hereby incorporated by reference in its entirety.

### FIELD OF TECHNOLOGY

This disclosure relates generally relates to glucose sensing and disease management systems.

### BACKGROUND

Diabetes is a chronic disease that impacts many individuals, both adults and children. The management of diabetes may include a measurement of glucose within the interstitial space including blood and/or interstitial fluid of a patient (herein a “user”) and, based on the measured glucose, administering insulin. A closed loop insulin administration system includes both a sensor to conduct glucose measurements from the interstitial space including blood and/or interstitial fluid of the user and an insulin administration device which administers insulin to the user based on the glucose measurements. Closed loop insulin administration systems allow individuals impacted by diabetes to go about daily life with much less worry about their insulin or glucose levels, which can vastly improve a diabetic's quality of life.

### SUMMARY

Various implementations of systems, methods, and/or devices within the scope of the appended claims each have several aspects, no single one of which is solely responsible for the desirable attributes described herein. Without limiting the scope of the appended claims, some prominent features are described herein.

Described herein is an analyte sensor transcutaneously inserted into the skin of the user which measures one or more analytes, such as glucose. The analyte sensor is an improvement over prior sensors in that the configuration of electrodes at varying depths within the skin of the user allows for a more accurate measurement of analyte at the injection site. Additionally, the plurality of electrodes increases the chances that proper wetting will occur and increases flexibility in the level of precision needed for placement of the electrodes at the injection site. In some implementations described herein, the electrode configuration allows for flexibility in the level of precision of layers of the sensor during manufacturing. At least some of the implementations described herein can be easily extended to allow for multi-analyte sensing, an additional improvement over prior sensor designs.

An analyte sensor can be configured to measure a concentration of at least one analyte at varying depths within a user. The analyte sensor can include a connector in electrical communication to a disease management system, and an elongated skin penetrating member having a length extending from a distal end to a proximal end, wherein the proximal end extends from the connector, the elongated skin penetrating member includes a semi-rigid substrate layer having a first side and a second side, at least one metal layer positioned on first side of the semi-rigid substrate layer including a plurality of working electrodes arranged along the length of the elongated skin penetrating member, at least one reference electrode, and at least one counter electrode, wherein the plurality of working electrodes includes at least one reference electrode therein, and wherein the plurality of working electrodes may be adjacent to the at least one counter electrode, and at least one insulation layer surrounding the at least one metal layer.

The analyte sensor can be further configured with one or more of the additional features as described herein. For example, the plurality of working electrodes can be in electrical communication with the connector. A voltage can be applied to the plurality of working electrodes to generate at least one measurement of the concentration of the at least one analyte. The second side of the semi-rigid substrate layer can further include at least one metal layer including a of the plurality of working electrodes, at least one reference electrode, and at least one counter electrode, wherein the plurality of working electrodes includes at least one reference electrode therein, and the plurality of working electrodes may be adjacent to the at least one counter electrode. The Analyte sensor can include four working electrode sets on the distal end of the elongated skin penetrating member on the first side, and four working electrode sets spanning a length of the elongated skin penetrating member on the second side. The plurality of working electrodes, the at least one reference electrode, and the at least one counter electrode can include platinum, gold, silver, silver chloride, rhodium, iridium, or a combination thereof. The elongated skin penetrating member can be configured to be at least partially implanted in a user. The elongated skin penetrating member can be configured to at least partially enter an adipose layer of the user. The elongated skin penetrating member can be configured to at least partially measure a concentration of an analyte at an adipose layer of the user. The elongated skin penetrating member can be configured to at least partially measure a concentration of an analyte at a dermis layer of the user. The analyte sensor can be configured to be disposed in a semi-permeable electrochemical cell.

Further, the plurality of working electrodes can include a series of individual working electrodes electrically connected and spaced equidistantly from each other. The series of individual working electrodes within the plurality of working electrodes can include one reference electrode. The series of individual working electrodes can include nine working electrodes. The plurality of working electrodes may be in physical contact with the at least one counter electrode.

An analyte measurement system for measuring a concentration of an analyte within a user an include an analyte sensor which can include a connector in electrical communication to a disease management system, an elongated skin penetrating member having a length extending from a distal end to a proximal end, wherein the proximal end extends from the connector, the elongated skin penetrating member can include a semi-rigid substrate layer having a first side and a second side, at least one metal layer positioned on the first side of the semi-rigid substrate layer including a plurality of working electrodes arranged along the length of the elongated skin penetrating member, at least one reference electrode, and an at least one counter electrode, wherein the plurality of working electrodes can include at least one reference electrode therein, and wherein the plurality of working electrodes is adjacent to the at least one counter electrode, and at least one insulation layer surrounding the at least one metal layer, a medication catheter configured to deliver medication to a user, a physiological sensor configured to communicate physiological values, an at least one communication components configured to transmit and receive information associated with the disease management system, and an at least one user interfacing component configured to accept and receive user input.

A method of measuring a concentration of at least one analyte in a user at an injection site can include inserting an elongated skin penetrating member having a plurality of working electrodes arranged along a length of the elongated skin penetrating member into an injection site of the user to contact a bodily fluid include at least one analyte, applying a voltage to the plurality of working electrodes wherein the plurality of working electrodes generate an electrical potential corresponding to a plurality of measurements of the at least one analyte at the injection site, communicating the plurality of measurements to a processor to determine a single analyte concentration, and reporting the single analyte concentration at the injection site to the user.

An analyte sensor to measure a concentration of at least one analyte at varying depths within a user can include a connector in electrical communication to a disease management system, and an elongated skin penetrating member having length extending from a distal end and a proximal end, wherein the proximal end extends from the connector, and wherein the distal end is an electrode well, wherein the electrode well includes a semi-rigid substrate layer having a first side and a second side, at least one metal layer positioned on the first side of the semi-rigid substrate layer including a plurality of working electrodes arranged along the length of the elongated skin penetrating member, at least one reference electrode, and at least one counter electrode, wherein the plurality of working electrodes includes at least one reference electrode therein, and wherein the at least one reference electrode, and the plurality of working electrodes are sequentially spaced along the electrode well, and at least one insulation layer surrounding the at least one metal layer.

The analyte sensor can include additional features as described herein. For example, the plurality of working electrodes can be in electrical communication with the connector. A voltage can be applied to the plurality of working electrodes to generate at least one measurement of the concentration of the at least one analyte. The plurality of working electrodes, the at least one reference electrode, and the at least one counter electrode can include of platinum, gold, silver, silver chloride, rhodium, iridium, or a combination thereof. The elongated skin penetrating member can be configured to be at least partially implanted in a user. The elongated skin penetrating member can be configured to at least partially enter an adipose layer of the user. The analyte sensor can be configured to be disposed in a semi-permeable electrochemical cell. The plurality of working electrodes can include a series of individual working electrodes electrically connected and spaced sequentially along the electrode well. The series of individual working electrodes within the plurality of working electrodes can include one reference electrode. The series of individual working electrodes can include three working electrodes. The proximal end of the elongated skin penetrating member can include a first narrowing portion such that a first cross-sectional area of the proximal end of the elongated skin penetrating member nearest to the connector may be greater than a second cross-sectional area of the proximal end of the elongated skin penetrating member closest to the distal end of the elongated skin penetrating member, and wherein the distal end of the elongated skin penetrating member includes a second narrowing portion, such that a first cross-sectional area of the distal end of the elongated skin penetrating member nearest to the connector may be greater than a second cross-sectional area of the distal end of the elongated skin penetrating member farthest from the connector. The plurality of working electrodes can include platinum. The reference electrode and/or the counter electrode can further include a composite material based on a silver/silver chloride composite. The counter electrode can further include platinum or gold.

A method of measuring a concentration of at least one analyte in a user at an injection site can include inserting an elongated skin penetrating member having a plurality of working electrodes arranged along the length of the elongated skin penetrating member at least into an injection site of a user, wherein the injection site includes of at least a dermis, an adipose, and at least one analyte of the user, applying a voltage to the plurality of working electrodes wherein the plurality of working electrodes generate an electrical potential corresponding to a plurality of measurements of the at least one analyte at the injection site, communicating the plurality of measurements to a processor to determine a plurality of analyte concentration values for at least the dermis and the adipose of the user, and reporting the plurality of analyte concentration values at the injection site to the user. Further, the plurality of working electrodes can include a series of individual working electrodes electrically connected and spaced equidistantly from each other. The series of individual working electrodes within the plurality of working electrodes can include one reference electrode. The series of individual working electrodes can include nine working electrodes.

### BRIEF DESCRIPTION OF THE DRAWINGS

These and other features, aspects, and advantages of the present application are described with reference to drawings of certain implementations, which are intended to illustrate, but not limit, the present disclosure. It is to be understood that the attached drawings are for the purpose of illustrating concepts disclosed in the present application and may not be to scale.

FIG. 1 illustrates an example disease management system that may be part of a disease management environment.

FIG. 2 illustrates an example disease management system that may be part of a disease management environment.

FIG. 3A illustrates an example of an implantable micro-electrochemical cell.

FIG. 3B is a block diagram of an example analyte sensor inserted within the skin of a user.

FIG. 4A illustrates an example implementation of an analyte sensor.

FIG. 4B illustrates an example electrode pattern for an analyte sensor.

FIG. 4C illustrates an example substrate that may be part of an analyte sensor.

FIG. 5A illustrates an example electrode well that may be part of an example analyte sensor.

FIG. 5B illustrates an exploded view of layers of an example analyte sensor.

FIG. 6A illustrates an example analyte sensor having narrowing portions.

FIG. 6B illustrates an exploded view of layers of an example analyte sensor.

FIG. 7 illustrates a workflow for analyzing concentration of an analyte at different depths of the skin as measured by a disease management system.

### DETAILED DESCRIPTION

Although certain example implementations are disclosed below, inventive subject matter extends beyond the specifically disclosed example implementations to other alternative implementations and/or uses and to modifications and equivalents thereof. Thus, the scope of the claims that may arise here is not limited by any of the particular example implementations described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain example implementations; however, the order of description should not be construed to imply that these operations are order dependent. Additionally, the structures, systems, and/or devices described herein may be implemented as integrated components or as separate components. For purposes of comparing various example implementations, certain aspects and advantages of these example implementations are described. Not necessarily all such aspects or advantages are achieved by any particular implementation. Thus, for example, various implementations may be carried out in a manner that achieves or optimizes one advantage or group of advantages as taught herein without necessarily achieving other aspects or advantages as may also be taught or suggested herein.

### Example Disease Management System

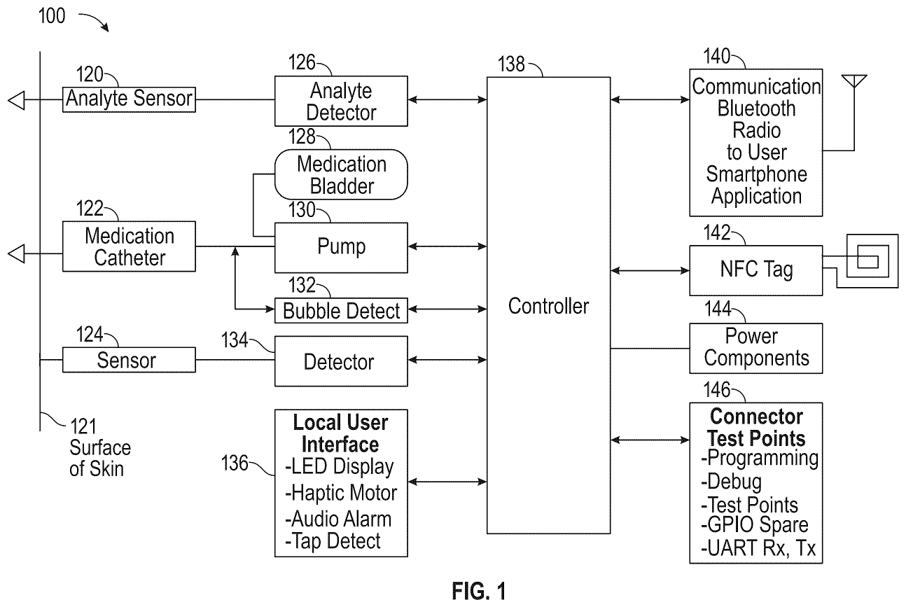

FIG. 1 illustrates a block diagram of a disease management system 100. The disease management system 100 may be part of a disease management environment, such as in the context of managing a disease such as diabetes or where continuous, regular, or periodic monitoring of a user is desired. A disease management environment can include use of a disease management system 100 for monitoring of a user's analytes for any number of reasons, including but not limited to, metabolic regulation, weight loss, or a combination thereof. While a disease management system 100 is referred to herein, the disease management system 100 may be used with or without the presence of disease or official diagnosis of such. While certain features are discussed herein with relation to a disease managements system 100, each and every feature may or may not be present and further features may be present not discussed herein. For example, a disease management system 100 may be described as having certain medication delivery mechanisms, such as a pump 130 and medication catheter 122, however, a disease management system 100 may or may not include these and/or associated features.

A disease management system 100 may be configured to measure one or more physiological parameters of a user (such as oxygen saturation, pulse rate, skin temperature, or other parameters), measure one or more analytes present in the blood of a user (such as glucose, lipids, or other analytes) and administer medication (such as insulin, glucagon, or other medication). The disease management system 100 may be configured to communicate with one or more hardware processors that may be external to the disease management system 100, such as a cloud based processor, a user device (such as a smart phone, smart watch, or tablet), health or clinician server devices, or any other connected device. A disease management system 100 may include various pieces of hardware to support authentication and pairing with another connected devices such as any of the user devices or connected devices described herein. The disease management system 100 may connect via Bluetooth communication with additional disease management systems or devices, and via Bluetooth communication with a paired user device running an associated control application. The disease management system 100 may connect to any of the devices mentioned herein via any other means such as via a Wi-Fi connection, a hardwired connection through a cable, or from a cellular network for example.

To support ease of use and safe interaction with the user, the system may incorporate user input through, for example, a tap-detecting accelerometer designed to detect user input and provide feedback via an audio speaker, haptic vibration, and/or optical indicators based on the received user input.

The system may be powered by one or more types of energy sources, for example a battery such as a lithium ion battery or any other type of battery source. The one or more energy sources may power and support both shelf-life and reliable operation once the disease management system 100 is applied to the skin of a user. Battery life may be managed through control of several planned levels of sleep and power consumption as determined by one or more processors (herein “a controller”).

As illustrated in FIG. 1, a disease management system 100 can include a controller 138. The controller 138 may be configured to communicate and/or control one or more components of the disease management system 100. For example, to support reliable operation of the disease management system 100, the controller 138 can be configured to monitor several system-health parameters, monitor temperatures of the included medication, and/or monitor ambient temperature for the life of the device. The controller 138 may include one or more hardware processors, such as a printed circuit board (PCB) or the like and memory. The controller 138 may be configured to communicate with peripheral devices or components to support the accurate measurement of physiological parameters and blood analytes, such as user pulse rate, temperature, blood oxygen saturation, and blood glucose, using detector electronics. The controller 138 may subsequently receive a signal from one or more peripheral devices or components, and calculate a dose or receive a calculated dose value from the one or more peripheral devices, and based on the calculated dose value, administer medication, such as insulin, by initiating actuation of an actuated pump. The controller 138 may record device activity, such as initiating actuation of an actuated pump, power on/off cycles, or any other device activity, and transfer the recorded data to non-volatile secure memory space within the controller 138 or external to the controller 138. At the end of the life of a device or system, the controller 138 can be configured to lock operation, wherein the controller 138 may protect the user's device activity data and other user data, and create a data recovery module as part of the controller 138 wherein a select third party, such as a designated healthcare professional or maintenance technician for example, may receive authenticated access to the recorded data.

A disease management system 100 may include an analyte sensor 120. The analyte sensor 120 may be configured to detect, for example, analytes in the user's blood. For example, an analyte sensor 120 can include a glucose sensing probe configured to pierce the surface of the skin 121 of a user. A disease management system 100 may include more than one of analyte sensor 120 to detect one or more analytes. An analyte sensor 120 may be configured to detect a plurality of analytes. Sensed analytes may include, but are not limited to, glucose, insulin, and/or other analytes. An analyte sensor 120 may be configured to communicate with an analyte detector 126, and/or with controller 138. The analyte detector 126 may be configured to receive a signal from one or more of analyte sensor 120 in order to measure one or more analytes in the blood of the user. The analyte detector 126 may be configured to communicate controller 138. For example, the analyte detector 126 may be configured to receive a sensed analyte from the analyte sensor 120, and transmit, to the controller 138 data representing a measured quantity of an analyte within the skin 121 of a user. Further the analyte detector 126 may receive, from the controller 138, control signals representative of one or more of an analyte to be detected.

A disease management system 100 may further include a medication catheter 122. The medication catheter 122 may be configured to administer medication, including, but not limited to insulin, to the user when the disease management system 100 is placed on the surface of the skin 121 of a user. The medication catheter 122 may be connected to a pump 130. The medication catheter 122 may receive medication from a pump 130. The pump 130 may be configured to cause medication to be administered to the user through the medication catheter 122. The pump 130 may include, but is not limited to, a pump such as described herein. The pump 130 may include an inlet attached to a medication bladder 128. The pump 130 may receive one or more types of medication from the medication bladder 128.

The medication bladder 128 may be configured to store medication to be administered. The medication bladder 128 may be configured to store medication for a prolonged period, such as 1 day, 3 days, 6 days, or more. The medication bladder 128 may be configured to store certain medication types, such as for example, insulin. A disease management system 100 may include one or more of medication bladder 128 connected to a pump 130 and/or connected to any other means of administering medication to a user. The one or more of medication bladder can include the same medications or any variation of different medications. A disease management system 100 may be configured to mix medications from one or more of medication bladder 128 prior to administration to the user.

A disease management system 100 may include a bubble detect sensor 132 configured to detect the presence of air bubbles in the medication prior to delivery to the user. The bubble detect sensor 132 may be in electrical communication with the controller 138. The bubble detect sensor 132 may be configured to sense whether air is present in the medication catheter 122 of the disease management system 100 or any other portion of the medication administration components of the disease management system 100 such as, for example, the pump 130 or the medication bladder 128. Additionally and/or alternatively, the bubble detect sensor 132 may transmit a signal to the controller 138, wherein the controller may determine whether air is present in one or more of the medication administration components of the disease management system 100.

A disease management system 100 may optionally include a physiological sensor 124. The physiological sensor 124 may include, for example, a pulse rate sensor, temperature sensor, and/or a pulse oximeter, or the like or a combination thereof. A disease management system 100 may be configured to include a plurality of physiological sensors. The physiological sensor 124 may be configured to communicate with a physiological detector 134. Additionally and/or alternatively, the physiological sensor 124 may be configured to communicate with the controller 138. The physiological detector 134 may be configured to receive a signals of the physiological sensor 124. The physiological detector 134 may be configured to measure or determine and communicate a physiological value from a received signal from a physiological sensor 124. The physiological detector 134 may be configured to transmit to the controller 138, a sensed physiological characteristic of the user. For example, the physiological detector 134 may be configured to transmit measured physiological values to the controller 138 and receive control signals from the controller 138. Alternatively and/or additionally, the physiological detector 134 may be part of, or configured as part of controller 138.

A disease management system 100 may further include one or more local user interfacing components 136. For example, a local user interfacing components 136 may include, but is not limited to, one or more optical displays, haptic motors, audio speakers, and/or user input detectors. An optical display may include one or more LED's configured to emit a plurality of different colored lights, or configured to emit light a pulsed light based on a mode or characteristic of the disease management system 100 as determined by controller 138. An optical display may include a digital display of information associated with the disease management system 100, including, but not limited to, device status, medication status, user status, measured analyte or physiological values, the like or a combination thereof. A user input detector may include an inertial measurement unit, tap detector, touch display, or other component configured to accept and receive user input. The one or more local user interfacing components 136 can include audio speakers. The audio speakers may be configured to communicate audible alarms related to device status, medication status user status, or any of the other device status or modes described herein. The controller 138 may be configured to communicate with the one or more local interfacing components 136 by, for example, receiving user input from the one or more user input components or transmitting control signals to, for example, activate a haptic motor, generate an output to the optical display, generate an audible output, or otherwise control one or more of the local user interfacing components 136.

A disease management system 100 may additionally include one or more communication components 140. Communication components 140 can include, but are not limited to, one or more radios configured to emit Bluetooth, cellular, Wi-Fi, or other wireless signals to communicate with an external device such as a medical or healthcare server, or a user device such as a smart phone or tablet. In some examples, communication components 140 can include a port configured for a wired connection. Additionally, a disease management system 100 may include an NFC tag 142 to facilitate communication with one or more internal or external hardware processors. The one or more communication components 140 which may include an NFC tag 142 may be configured to communicate with the controller 138 in order to transmit and/or receive information associated with the disease management system 100. For example, a controller 138 may communicate medication information, measured user physiological data, measured analyte data, and any other data through the one or more communication components 140 to an external device such as any of the external devices described herein. Additionally and/or alternatively, the communication components 140 may receive, from an external device, and transmit to the controller 138, instructions associated with an operation of the disease management system 100, for example, the configuration of measurement sampling rates, medication delivery, or other information associated with operation of the management system 100.

A disease management system 100 may include one or more power components 144. The one or more power components 144 may include one or more power sources, such as for example, one or more batteries. Additionally, and/or alternatively, the power sources can include power management components such as for example, a voltage regulator. Power from the one or more power components 144 may be accessed by the controller and/or other components of the disease management system 100 to operate the disease management system 100.

The controller 138 may be configured to further regulate power consumption of the disease management system 100 by entering one or more of modes of operation. The modes of operation can include at least a power mode and a sleep mode, to help regulate power usage. For example, a disease management system 100 may include a sleep mode, wherein the controller 138 is configured for minimal functions, which may include only operating such functions of the controller 138 such as the real time clock (RTC) and alarms to wake the system and generate a temperature measurement of the system, or the like. In another example, a disease management system 100 may include a measure temperature mode which may correspond to a low power mode with reduced functions. The measure temperature mode may be triggered by the RTC where the system is configured to generate a temperature measurement, save the value in memory, and return the system to a sleep mode. In another example, a disease management system 100 may include a wake up mode. The wake up mode may be triggered by, for example an external communication received by the NFC tag 142 and/or the communication components 140, and allow the system to pair with an external device with, for example, via Bluetooth. Additionally and/or alternatively, the wake up mode may be triggered internally by the controller 138. If a pairing event does not occur, the system may return to sleep mode.

In another example, a disease management system 100 may include a pairing mode. The pairing mode may be triggered by for example, the NFC tag 142 and/or communication components 140. When a received signal from an external device is recognized by the controller 138, the controller 138 may proceed to pair with the application and set the disease management system 100 to an “ON” condition and communicate to, for example, the cloud or other external device to establish initial data movement.

In another example, a disease management system 100 may include a rest mode where the controller 138 is configured to enter a lower power mode between measurements, for example a physiological measurement by the physiological sensor 124 and/or by the analyte sensor 120. In another example, a disease management system 100 may include a data acquisition mode where the system is configured to enter a medium power mode where data may be transferred to an external device and/or instructions may be received by the controller 138. In another example, a disease management system 100 may include a parameter calculation mode where the controller 138 is configured to enter a medium power mode where parameter calculations, such as a blood glucose calculations, are performed and data is communicated to an external device such as, for example a medical or healthcare server and/or the cloud. In another example, a disease management system 100 may include a pump mode where the system is configured to enter a higher power mode where the pump is energized to deliver medication to the user.

A disease management system 100 may include one or more connector test points 146. The connecter test points may be configured to aid in programming, debugging, testing or other accessing of the disease management system 100. The one or more connector test points 146 may include, for example, a GPIO spare, UART receiver or transmitter, the like or a combination thereof. The connector test points may be, for example, electrically connected to the controller 138, and/or any other component of the disease management system 100.

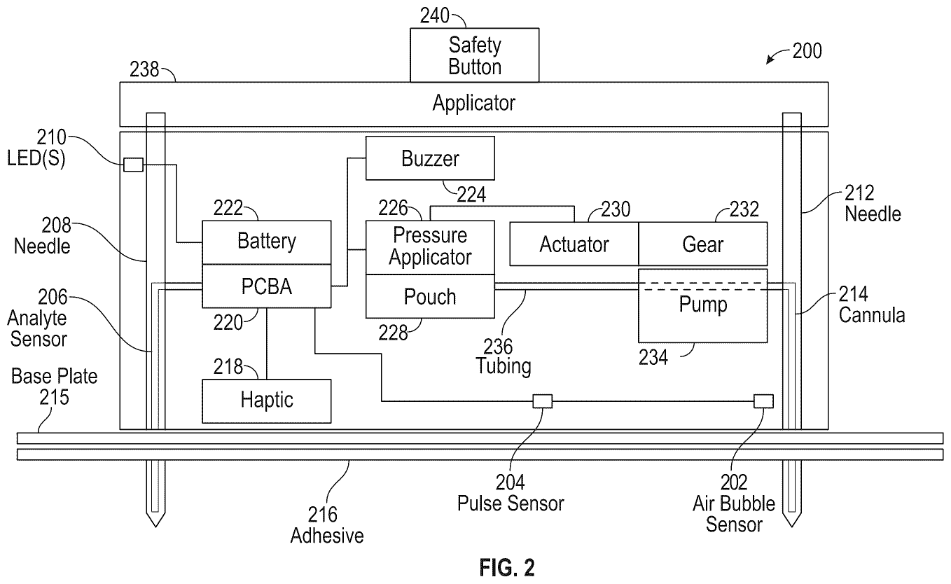

FIG. 2 illustrates an example implementation of a disease management system 200 and applicator 238 that may be applied to the skin of a user. Disease management system 200 can include any one or more of the features discussed above with respect to the disease management system 100 in addition to the features described below. In the illustrated example, an applicator 238 may be configured to mate with the disease management system 200. However, a disease management system 200 may or may not include a separate applicator 238. An applicator 238 may include a safety button 240 for release or other interaction with the applicator 238. In the illustrated example, a disease management system 200 may include one or more LEDs 210 that may be configured to output information using one or more of color, frequency, and/or length of display. The disease management system 200 may include a buzzer 224, haptic actuator 218, or other feedback mechanism, such as a speaker to output information to the user, such as an alarm. A disease management system 200 may include a battery 222, and/or a controller 220. A disease management system 200 may include aspects of a medication administration system, such as a bladder 228, and bladder pressure applicator 226. The bladder pressure applicator 226 may provide pressure on the bladder 228. The disease management system 200 may further include an actuator 230, pump gears 232, tubing 236 and/or a pump 234. A disease management system 200 may include one or more needles 208 that may include one or more of analyte sensor 206. The analyte sensor 206 can be, for example, a glucose sensor. A disease management system 200 may include one or more medication needles 212 that may include one or more of cannula 214 configured to administer medication to the user. A disease management system 200 may include an air bubble sensor 202 configured to detect the presence of air bubbles in the medication prior to delivery to the user. A disease management system 200 may include one or more physiological sensors 204, such as, for example, a non-invasive physiological sensor including but not limited to a pulse sensor, SpO2 sensor, Oxygen saturation sensor, and/or a temperature sensor. The disease management system 200 may include a base plate 215 and an adhesive layer 216 below the base plate 215 to provide adhesion of the disease management system 200 to the user's skin. A housing of the disease management system 200 may consist of a combination of flexible and rigid material so as to both provide support for the components of the disease management system 200 and allow conforming, at least in part, of the disease management system 200 to the skin of the user.

The adhesive layer 216 may be configured to provide adhesion for a prolonged period. For example, the adhesive layer 216 may be configured to adhere the disease management system 200 to the skin of a user for a period of 1 day, 3 days, 6 days, or more or fewer days or hours. The adhesive layer 216 may be configured to have an adhesive force sufficient to prevent accidental removal or movement of the disease management system 200 during the intended period of use of the disease management system 200. The adhesive layer 216 may be a single layer of adhesive across at least a portion of a surface of the disease management system 200 that is configured to interface with the user. The adhesive layer 216 may include a plurality of adhesive areas on a surface of the disease management system 200 that is configured to interface with the user. The adhesive layer 216 may be configured to be breathable, and/or adhere to the user's skin after wetting by humidity or liquids such as tap water, saltwater, and/or chlorinated water. A thickness of the adhesive may be, for example, in a range of 0.1 to 0.5 mm or in a range of more or less thickness.

In some examples, one or more needles 208, and/or a cannula 214 may be inserted at different depths into the skin of a user based on a user's age, weight, and/or other any other characteristics of the user. For example, a depth of insertion of a medication cannula 214 may be approximately 3 mm for a user who is approximately 7 to 12 years old. In another example, a depth of insertion of a medication cannula 214 may be approximately 4 mm for a user who is approximately 13 or more years old. In another example, a depth of insertion of a medication needle 212 may be approximately 4 to 4.5 mm for a user who is approximately 7 to 12 years old. In another example, a depth of insertion of a medication needle 212 may be approximately 5 to 5.5 mm for a user who is approximately 13 or more years old. In another example, a depth of insertion of an analyte sensor 206 may be approximately 3 mm for a user who is approximately 7 to 12 years old. In another example, a depth of insertion of an analyte sensor 206 may be approximately 4 mm for a user who is approximately 13 or more years old. In another example, a depth of insertion for a needle 208 associated with an analyte sensor 206 may be approximately 4 to 4.5 mm for a user who is approximately 7 to 12 years old. In another example, a depth of insertion for a needle 208 associated with an analyte sensor 206 may be approximately 5 to 5.5 mm for a user who is approximately 13 or more years old. However, other values or ranges for any of the inserted components are also possible.

An analyte sensor such as analyte sensor 120, and/or 206, as described in FIGS. 1-2, or any of the analyte sensors described herein can be configured to at least partially implant into the skin tissue of the user. The analyte sensor may include one or more sensor components or electrodes enclosed at least in part in a permeable cell. In some examples, the analyte sensor and/or one or more portions of the analyte sensor may not be enclosed in a permeable cell. The permeable cell may include one or more permeable portions configured to allow passage of analyte including fluid from the surrounding tissue of the user to a portion of the permeable cell including the one or more sensor components. The one or more sensor components or electrodes may be configured to measure at least one analyte, such as glucose or other analyte present at the tissue site of the user.

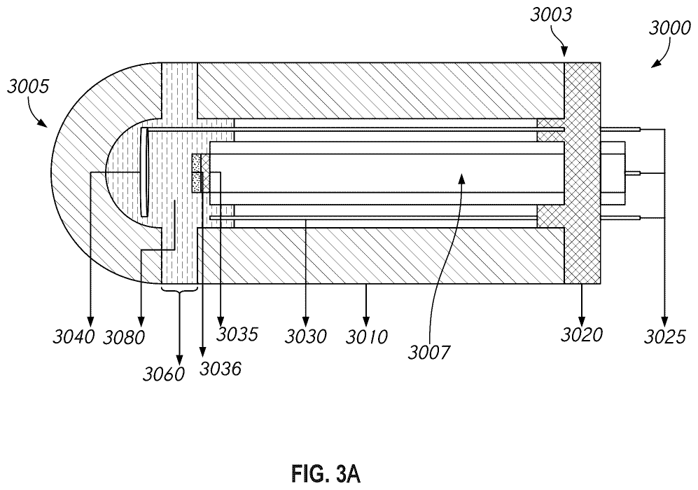

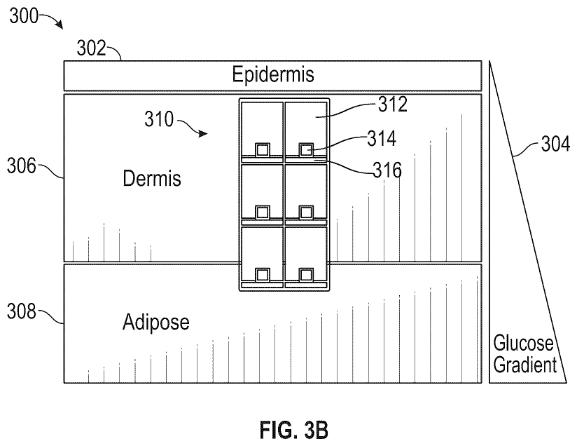

FIG. 3A illustrates an example of an implantable micro-electrochemical (or permeable) cell 3000 which may be associated with an analyte sensor, such as described herein. U.S. patent application Ser. No. 18/050,401 filed Oct. 27, 2022, titled “IMPLANTABLE MICRO-ELECTROCHEMICAL CELL” is hereby incorporated by reference in its entirety. The implantable micro-electrochemical cell may be implanted at least in part under the skin of the human body. The implantable micro-electrochemical cell can vary in depth of which it is implanted under the skin. For example, the implanted micro-electrochemical cell can be anywhere between up to and including 100% implanted underneath the skin. In some examples, the cell 3000 may be implanted so that at least a permeable sidewall portion may be implanted below the skin. In some examples, the cell 3000 may be implanted at a depth such that the permeable sidewall portion is in contact with bodily fluid containing analytes. In some instances, the implantable micro-electrochemical cell 3000 may be injected or inserted into the human body using a needle, such as described above (for example, with reference to FIG. 2), and connect to a device, such as a combined glucose sensor and insulin pump, that is located outside of the human body. In some example, one or more elements or substances inside the implantable micro-electrochemical cell 3000 may be at least partially protected from the outside. The implantable micro-electrochemical cell 3000 may be configured to prevent contact of at least some of the interior components of the cell with portions of the tissue of the patient. In some examples, the cell 3000 may include a smooth outer surface. Advantageously, the smooth outer surface of the cell may protect tissue surrounding the outer surface of the cell 3000 from being irritated or injured from contact with the cell 3000. In some examples, the smooth outer surface may be made of, for example, silicones, polyethylene, polyimide, or the like. Furthermore, the longevity of the cell 3000 may be enhanced from reduced friction or other contact with tissue of the patient. In some examples, the cell 3000 may be coated with a material to enhance biocompatibility upon implantation of the cell.

The implantable micro-electrochemical cell may include a permeable cell. The permeable cell may include a component that is at least partially closed to outside material. The geometry of the permeable cell may include a three dimensional shape having at least one smooth surface, such as a cuboid, a pyramid, a cylinder, or other object with at least one flat or curvilinear geometric shape or side. In some examples, the permeable cell shape may include a container portion 3010. The container portion 3010 may be tubular shape, such as illustrated in FIG. 3B. A tubular shape for the permeable cell may provide greater surface area for the sensors compared to a flat surface. Additionally, a tubular shape may be easier to manufacture than a different shape, for example rectangular. Furthermore, a tubular shape may ease implantation of the permeable cell, such that a tubular shape may increase comfortability of the patient and/or a tubular shape may achieve a greater implantation depth than a shallower shape.

The permeable cell may be at least partially composed of a biocompatible material, such as a biocompatible plastic or the like. For example, the permeable cell may include at least one layer of a polyamide, other polymer, or the like. In some examples, the container portion 3010 may have a length between approximately 3 mm to approximately 8 mm, such as 5 mm, an outer diameter between approximately 100 micrometers to approximately 500 micrometers, such as 300 mm, and an inner diameter between approximately 150 micrometers to approximately 450 micrometers, such as 250 mm. However, the cell may have smaller or larger dimensions. In some examples, preferably, the container portion 3010 may have a length of approximately 5 mm. In some examples, preferably, the container portion 3010 may have an outer diameter of approximately 300 micrometers. In some examples, preferably, the container portion 3010 may have an inner diameter of approximately 250 micrometers. Advantageously the diameter of the cell may be sufficiently small so as to reduce the likelihood of a painful insertion or implantation of the micro-electrochemical cell in the tissue of the patient. Additionally, in some cases, the diameter of the cell may be sufficiently small to reduce damage or injury caused by the implantation of the cell. Furthermore, the overall size of the cell 3000 may be designed or sized to simplify the implantation procedure, such that the need for bulkier, less user friendly, tools may be minimized or eliminated.

The permeable cell 3000 may have at least one open end 3003 and a closed end 3005. The permeable cell may be sealed at the open end 3003 by a seal 3020. The seal 3020 may be used to close off the open end 3003 of the permeable cell 3000 or container portion 3010 to prevent elements of the container portion 3010 and substances within the container portion 3010 from being misplaced. Additionally, the seal 3020 may be configured to include at least one electrical feedthrough to allow for electrode leads 3025 to pass through the seal 3020 and make an electrical connection with components of the at least one analyte sensor within the permeable cell. The electrode leads 3025 may then be configured to connect to one or more electrical components of a connected device, such as a combination insulin pump and analyte sensor and/or disease management device described above (for example, with reference to FIG. 2). In some examples, the electrical feedthrough may at least partially not be implanted in the patient. This may provide easy access to the electrode without having to remove the permeable cell.

The open end 3003 may be located at the top portion of the permeable cell 3000. The closed end 3005 may be located at the bottom portion of the permeable cell 3000. In some examples, the open end 3003 may be positioned outside of the patient's body. In some examples, the closed end 3005 may be positioned inside of the patient's body. The open end 3003 positioned outside of the patient's body can provide a user access to the internal compartment of the permeable cell 3000 without having to remove the permeable cell 3000 from the patient's body. This can minimize the number of times the permeable cell 3000 may be implanted and/or reimplanted after the initial implantation of the permeable cell 3000. Additionally, the open end 3003 positioned outside of the patient's body and the closed end 3005 positioned inside of the patient's body can help to ensure that the internal elements of the permeable cell 3000 remain inside the permeable cell 3000.

In some examples, the closed end 3005 may be curved. This can increase the comfortability of the patient. For example, a curved closed end 3005 can help to ensure no sharp edges come in contact with the tissue site. This may minimize the irritation that the tissue site may experience.

An implantable micro-electrochemical cell 3000 may include one or more physiological sensors, including but not limited to an analyte sensor 3007. An analyte sensor 3007, such as a glucose sensor, may include an amperometric electrochemical biosensor generating a current from the electrochemical reaction between an analyte, such as glucose and a glucose oxidase layer on a working electrode. An analyte sensor 3007 may include some combination of electrodes connected to one or more electrode leads 3025. For example, an analyte sensor 3007 may include at least one electrode, such as at least one reference electrode 3030, at least one counter electrode 3040, and/or at least one working electrode 3035. Furthermore, in some examples, the at least one electrode may include a non-electrochemical electrode or other sensor configured to measure a physiological parameter of a patient (e.g., optical sensor). The one or more electrodes may be rectangular in shape. In other examples, the one or more electrodes may be cylindrical, conical, triangular, etc. in shape. In another example, the shape of the electrodes can include one or more of the shapes described herein and is not necessarily limited to the shape as defined and illustrated of FIG. 3A.

The one or more working electrodes 3035 may be used to measure the presence and/or amount of the analyte within the bodily fluid. The reference electrode 3030 may have an accurately maintained potential to be used as a reference to other working electrodes 3035 within the cell. The counter electrode 3040 may act as a reference half-cell to supply the required current to the working electrode 3035 for the electrochemical reaction. The one or more electrodes may be positioned in the center of the cell 3000. This may increase the ability for the one or more electrodes to measure analytes at the tissue site by allowing the bodily fluid to come in contact with all sides of the electrodes. In some aspects, one or more of the electrodes may include one or more metals. In some aspects, at least one electrode may include platinum (Pt), gold (Au), silver (Ag), rhodium (Rh), iridium (Ir), or combinations thereof.

The working electrode 3035 may have a tip 3036 that is modified with a suitable sensing element (e.g., an enzyme or the like). In some examples, the working electrode 3035 may contain an insulting layer with a thickness of between approximately 10 micrometers to approximately 20 micrometers, such as 15 mm. In some examples, preferably, the insulating layer may have a thickness of approximately 15 micrometers. The working electrode 3035 may have a diameter of approximately 80 micrometers to 120 micrometers. In some examples, the working electrode 3035 may have a diameter of approximately 100 micrometers.

In some examples, the one or more working electrodes may include nanomaterials, polymers and/or polymeric composites such as chitosan, cellulose, and conducting polymers. In one aspect, the working electrode 3035 may include Pt. In some aspects, the working electrode 3035 may include both Pt and Au. In another aspect, the working electrode 3035 includes both Pt and Ir. In some aspects, the working electrode 3035 may include a top layer and a bottom layer. In some aspects, the bottom metal layer of a working electrode 3035 may be at least one of Au, Ag, or Pt, and the top metal layer of the working electrode 3035 may be at least one of Au, Ag, or Pt. In some aspects, the thickness of the bottom metal layer of the working electrode 3035 may be about 2 μm, about 2.5 μm, about 3 μm, about 3.5 μm, about 4 μm, or any other thickness. In some aspects, the thickness of the top metal layer of the working electrode 3035 may be about 50 Å, about 70 Å, about 90 Å, about 100 Å, about 120 Å, about 150 Å, or any other thickness. In some aspects, the thickness of the top metal layer of the working electrode 3035 may be much less than the thickness of the bottom metal layer. The ratio of the thickness of the top metal layer to the thickness of the bottom metal layer of the working electrode 3035 may be less than about 1/500, 1/300, 1/100, or less than any other ratio.

An analyte sensor 3007 may include one or more reference electrodes 3030. The reference electrode 3030 of the analyte sensor may have a diameter of approximately 10 micrometers to 30 micrometers. In some examples, the diameter of the analyte sensor 3007 may be approximately 20 micrometers.

The one or more reference electrodes 3030 may include silver. In some aspects, the reference electrode may include Ag/AgCl, or the like. In one embodiment, the reference electrode 3030 may include silver and silver chloride (Ag/AgCl), Hydrogen, SCE, or the like. In some aspects, the thickness of the metal layer in the reference electrode 3030 may be about 2 μm, about 2.5 μm, about 3 μm, about 3.5 μm, about 4 μm, or any other thickness. In some aspects, the thickness of the reference electrode 3030 may be about 2 μm, about 2.5 μm, about 3 μm, about 3.5 μm, about 4 μm, and or other thickness. In some aspects, the reference electrode 3030 may include silver. In some aspects, the reference electrode 3030 may include of a bottom metal layer and a top metal layer. In some aspects, the bottom metal layer of the reference electrode 3030 may be about 2 μm, about 2.5 μm, about 3 μm, about 3.5 μm, about 4 μm, and or other thickness. In some aspects, the reference electrode 3030 may include a top metal layer. The top metal layer of the reference electrode 3030 may be about about 70 Å, about 90 Å, about 100 Å, about 120 Å, about 150 Å, or any other thickness. In some aspects, the thickness of the working electrode 3035 and the reference electrode 3030 may be similar.

An analyte sensor 3007 may include one or more counter electrodes 3040. The counter electrode 3040 may have a diameter of approximately 10 micrometers to 40 micrometers. In some examples, the counter electrode 3040 may have a diameter of approximately 30 micrometers. In some aspects, the thickness of the metal layer in the counter electrode may be about 2 μm, about 2.5 μm, about 3 μm, about 3.5 μm, about 4 μm, and or other thickness.

The counter electrode 3040 may include one or more metals as described herein. In one embodiment, the counter electrode 3040 includes Au. In another embodiment, the counter electrode 3040 includes Pt. In another embodiment, the counter electrode 3040 includes carbon.

The at least one electrode lead 3025 may be sealed, such that an inner side of the container portion 3010 is isolated from the at least one electrode leads 3025. In some examples, the at least one electrode lead 3025 may be sealed with a polymer resin or the like.

The implantable micro-electrochemical cell 3000 may include at least one porous interface 3060. The at least one porous interface 3060 may include at least a portion of the permeable cell 3000 that is configured to allow at least some transmission of analytes at the implantation site of the cell to access an interior portion of the cell 3000 and make contact with an analyte sensor 3007 inside the interior portion. In some examples, a porous interface 3060 may be a mesh or other permeable membrane. In some examples, the porous interface 3060 may be generated by laser drilling the container portion 3010 so as to create a plurality of holes or pores in the surface of the container portion 3010. In other examples, the container portion 3010 may be formed with a mold and polymer resin to encompass the porous interface 3060. The diameter of pores or holes in the porous interface 3060 may range between 10-50 micrometers or more or less than that range. In some examples, the porous interface 3060 may be adequately sizable enough to allow for diffusion of glucose into a fluid medium 3080 within the cell with minimal resistance, which may increase the ability for fresh glucose or other analytes to enter the cell 3000 from surrounding tissue of the patient when the cell 3000 is implanted.

The working electrode 3035 may be configured to measure analytes within a fluid medium 3080. The fluid medium 3080 may be provided into the interior of the permeable cell at the site of the porous interface 3060. The fluid medium 3080 may be configured to act as an interface and provide fluid communication with bodily fluid or analytes entering the permeable cell 3000 through the porous interface 3060 and at least a portion of the at least one analyte sensor 3007. The fluid medium 3080 may contain a cross-linked water absorbing polymer matrix. The cross-linked water absorbing polymer matrix may include a hydrogel formed by cross-linking polyethylene glycol diglycidyl ether and polyethylene glycol diamine.

### Example Analyte Sensor Systems and Electrode Composition

FIG. 3B. illustrates a block diagram of an example analyte sensor 300 of a disease management system, such as the disease management system of 100 or 200 as illustrated in FIGS. 1 & 2, inserted within the skin of a user. The analyte sensor may include a plurality of electrode groupings 310. The electrode groupings 310 may include electrode groupings to facilitate measurement of glucose at one or more electrodes within the electrode grouping 310. The electrode groupings 310 may include a matrix of one or more electrodes. The one or more electrodes of the electrode groupings 310 may include a plurality of counter electrodes 312, a plurality of working electrodes 314, and a plurality of reference electrodes 316. In another aspect, an electrode grouping 310 may be one counter electrode 312, one working electrode 314, and one reference electrode 316.

In some aspects, a counter electrode 312 within the grouping 310 can have a width of about 0.05 to about 2 mm. In some aspects the width of the counter electrode 312 is approximately 0.485 mm. In some aspects, a counter electrode 312 within the grouping 310 can have a height of about 0.025 to about 1 mm. In some aspects the height of the counter electrode 312 is approximately 0.695 mm.

In some aspects, a working electrode 314 within the grouping 310 can have a width of about 0.05 to about 1 mm. In some aspects the width of the working electrode 314 is approximately 0.25 mm. In some aspects, a working electrode 314 within the grouping 310 can have a height of about 0.05 to about 1 mm. In some aspects the height of the working electrode 314 is approximately 0.25 mm.

In some aspects, a reference electrode 316 within the grouping 310 can have a width of about 0.05 to about 2 mm. In some aspects the width of the reference electrode 316 is approximately 0.485 mm. In some aspects, a reference electrode 316 within the grouping 310 can have a height of about 0.025 to about 0.2 mm. In some aspects the height of the reference electrode 316 is approximately 0.095 mm.

The plurality of electrode groupings 310 may be arranged in a configuration to allow measurement of glucose at a plurality of depths of tissue. For example, the configuration may include a matrix or grid. The grid may include a 2×2, 2×3, 2×4, 2×5, 1×2, 1×3, 1×4, 1×5 matrix or any other matrix configuration. The configuration may include two or more of the electrode groupings 310 at the same depth such that if one electrode grouping of the electrode groupings 310 fails, another electrode grouping 310 at the same depth may be used to measure an analyte at that depth. The grid may be long enough in length to allow for contact of at least a portion of the electrode groupings 310 with dermis tissue and at least a portion of the electrode groupings 310 to be in contact with adipose tissue. For example, as illustrated in FIG. 3B, the analyte sensor 300 may at least partially enter the epidermis 302, the dermis 306 and/or the adipose 308 of a user.

In some aspects of the analyte sensor 300 of FIG. 3B, the width of a 2×3 electrode grouping 310 may be approximately 0.5 to 2 mm. In some aspects the width of a 2×3 electrode grouping 310 is about 1 mm. In some aspects, the height of a 2×3 electrode grouping 310 may be approximately 1 to 10 mm. In some aspects the height of the 2×3 electrode grouping 310 is about 2.5 mm.

Advantageously, the configuration of electrode groupings 310 at differing depths allows for more complex and potentially more accurate measurement of analyte, such as glucose than with an analyte sensor configured to measure at a single or similar depths. For example, a concentration of glucose in a patient's tissue varies with depth. This variation can be referred to as a glucose gradient 304. As illustrated in FIG. 3B, a glucose gradient 304 of a user's skin increases as the electrodes penetrate deeper into the user's skin. Thus, a glucose gradient 304 is elevated for the electrodes within grouping 310 located at or near the adipose 308. As electrodes within grouping 310 span the different layers of the skin, a disease management system may allow for several measurements at several depths within the skin of a user. The varying depths of electrode groupings 310 and corresponding measurements allows for differential measurements of an analyte. The inclusion of multiple measurements at more than one depth within the skin of a user helps reduce inaccuracies and human error that may be caused when a medical professional inserts an analyte sensor into the skin of a user.

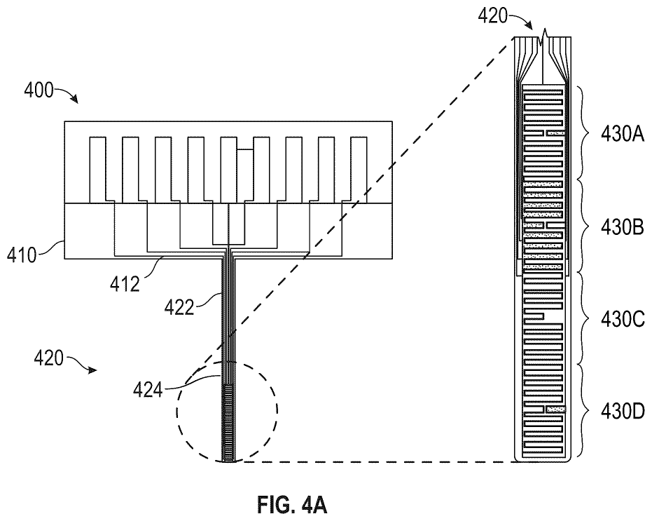

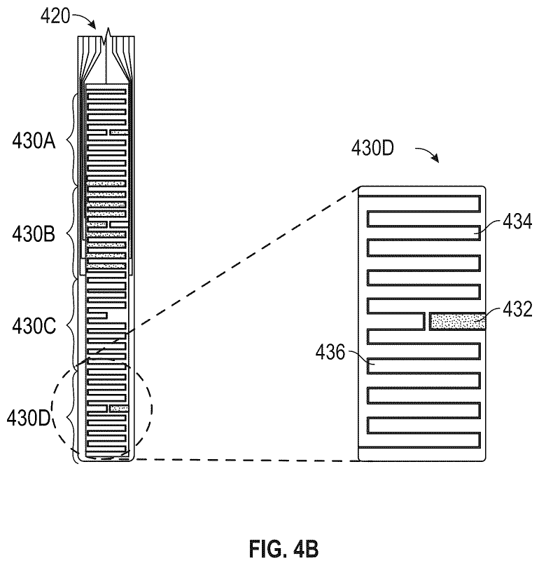

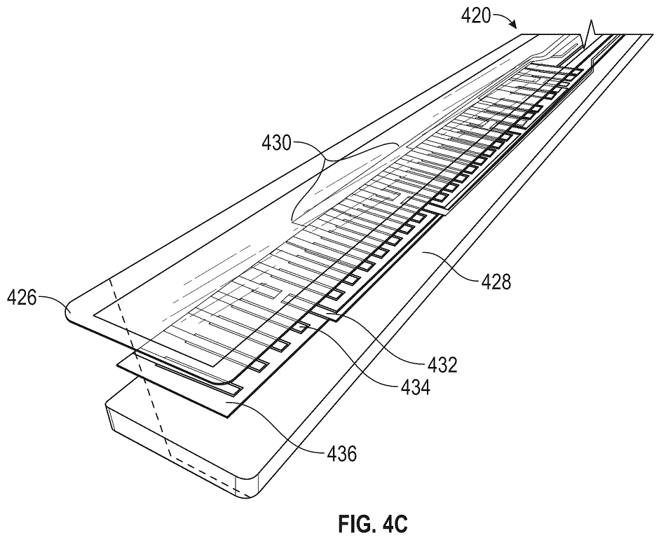

FIGS. 4A-C illustrate an example of an analyte sensor 400. The analyte sensor 400 may be similar to and/or the same as analyte sensor 120, 206, and/or 300 as illustrated in FIGS. 1-2, and 3B respectively, and/or another analyte sensor as described herein. Analyte sensor 400 may be implanted into the skin of a user. In some instances, the analyte sensor 400 may be injected or inserted into the skin of a user using a needle. In one example, one or more elements or substances inside the analyte sensor 400 may be protected from the ambient environment. For example, the analyte sensor 400 can be configured to prevent contact of at least some of the interior components of the analyte sensor with portions of the tissue of the user. The analyte sensor 400 may include a smooth outer surface. Advantageously, the smooth outer surface of the analyte sensor may protect tissue surrounding the outer surface of the analyte sensor 400 from being irritated or injured from contact with the analyte sensor 400. Furthermore, the longevity of the analyte sensor 400 may be enhanced from reduced friction or other contact with tissue of the user.

The analyte sensor 400 may be connected to a device via connector 410, such as a combined glucose sensor and insulin pump, and/or a controller such as controller 138 of FIG. 1 or another device, such as a continuous glucose monitor (CGM). The connector 410 may be configured to facilitate electrical communication from the analyte sensor 400 to a controller, such as a controller 138 described above. Connector 410 may be located outside the skin of the user. The connector 410 may provide communication from one or more electrodes of the analyte sensor 400 via electrode leads 412. The electrodes may then be configured to connect to one or more electrical components of a connected device, such as the controller and/or other components as described in FIGS. 1 and/or 2, and communicate to the connected device, a measurement of an analyte concentration within the skin of a user.

Additionally, the analyte sensor 400 may include a top portion 422 that extends from the connector 410. The analyte sensor may include a bottom portion 424 that further extends from the top portion 422. The bottom portion 424 of the analyte sensor 400 may include an elongated skin penetrating member 420 fixedly attached to the bottom portion 424. The elongated skin penetrating member 420 may be configured to at least partially implant into the tissue of the patient. For example, the elongated skin penetrating member 420 may be configured to at least partially implant into the epidermis, dermis, and/or adipose of the patient, such as illustrated in FIG. 3B. The geometry of the elongated skin penetrating member 420 may include a three dimensional shape having at least one smooth surface, such as a cuboid, a pyramid, a cylinder, or other object with at least one flat or curvilinear geometric shape or side. Additionally, the elongated skin penetrating member 420 may be flat with two smooth surfaces, such as a square or a rectangle. The elongated skin penetrating member 420 may be configured to include at least one electrical feedthrough to allow for electrode leads 412 to pass through and make an electrical connection with components of the connector 410.

Additionally, the analyte sensor 400 may be at least partially composed of a biocompatible material, such as a biocompatible plastic or the like. For example, the analyte sensor 400 may include at least one layer of a polyamide, other polymer, or the like. In some aspects a portion of the elongated skin penetrating member 420 may extend about 1 to about 5 mm from the bottom portion 424. In some aspects, a portion of the elongated skin penetrating member 420 may extend approximately 2.2 mm from the bottom portion 424. In some aspects, a portion of the elongated skin penetrating member 420 may be about 0.03 to 1 mm wide. In some aspects, a portion of the elongated skin penetrating member 420 is approximately 0.34 mm wide. In some aspects, the length of the connector 410, top portion 422, bottom portion 424 and skin penetrating member 420 is about 5 to about 20 mm in total length. In some aspects, the length of the connector 410, top portion 422, bottom portion 424, and skin penetrating member 420 is approximately 10 mm in length. Advantageously the width of the elongated skin penetrating member 420 may be sufficiently small so as to reduce the likelihood of a painful insertion or implantation in the tissue of the user.

FIG. 4A-B illustrate an example layout of electrode sets 430 A-D on a skin penetrating member 420. The analyte sensor 400 may include some combination of electrodes connected to electrode leads 412. For example, electrode sets 430A-D may be spaced equidistantly on the elongated skin penetrating member 420. Additionally and/or alternatively, the electrodes may be placed on the top portion 422, and/or any other portion of the elongated skin penetrating member 420. In one example implementation, there may be four of electrode sets 430A-D aligned down the elongated skin penetrating member 420. Within each of electrode sets 430A-D, there may be a reference electrode 432, a counter electrode 436, and/or a working electrode 434. The working electrode 434 may be configured to measure one or more analytes in fluid at the tissue site of the user. In some aspects, the area per working electrode 434 may be about 0.01 mm² to about 0.1 mm². In some aspects, the area per working electrode 434 is approximately 0.038 mm². In some aspects, the total area of all working electrodes 434 on located on one side of the elongated skin penetrating member 420 is about 0.1 mm² to about 1 mm². In some aspects, the total area of all working electrodes 434 located on one side of the elongated skin penetrating member 420 is approximately 0.153 mm². Additionally and/or alternatively, electrodes 432, 434, and/or 436 may be located on at least two sides of the elongated skin penetrating member 420. In some aspects, the total area of all working electrodes 434 on located on two sides of an elongated skin penetrating member 420 is about 0.1 mm² to about 1 mm². In some aspects, the total area of all working electrodes 434 located on two sides of the elongated skin penetrating member 420 is approximately 0.3.06 mm².

FIG. 4C illustrates an example implementation of a skin penetrating member 420 with electrodes 432, 434, and/or 436 and a layer of insulation 426 at the elongated skin penetrating member 420 of an analyte sensor 400. In some examples, four or more of electrode set 430A-D may be enclosed by insulation 426 on a semi-rigid substrate 428. The one or more of working electrode 434, and/or counter electrode 436 may be spaced among a metal layer of the elongated skin penetrating member 420. In some example implementations, the reference electrode 432 may consist of Ag/AgCl. In some examples the counter electrode 436 may consist of platinum. Furthermore, in some example implementations, the elongated skin penetrating member 420 may include a non-electrochemical electrode such as for example, an optical sensor.

In some implementations, the analyte sensor 400 may include one or more of electrodes 432, 434, and/or 436 on both sides of the elongated skin penetrating member 420. The one or more of electrodes 432, 434, and/or 436 located on both sides of the skin of the penetrating member 420 can be configured to generate two or more times as many sample measurements of an analyte at an injection site of a user in comparison to electrodes 432, 434, and/or 436 located on only one side of the elongated skin penetrating member 420. Advantageously, having one or more electrodes 432, 434, and/or 436 on both sides of the skin of the penetrating member 420 may result in more accurate reporting of concentrations of an analyte within the skin of a user.

In an example implementation, the analyte sensor 400 can include electrode sets 430A-D as part of skin penetrating member 420. Electrode sets 430A-D can include at least one or more of a reference electrode 432, a working electrode 434, and/or a counter electrode 436. The electrodes 432, 434, and/or 436 can be fixedly attached to the elongated skin penetrating member 420 of an analyte sensor 400, as illustrated in FIG. 4C. For example, each electrode set 430A-D can include at least one reference electrode 432 positioned along a first end of the elongated skin penetrating member 420 such that the reference electrode 432 contacts a surface of the elongated skin penetrating member 420 that extends from the first end towards an opposite end of the elongated skin penetrating member 420. The contact surface of the reference electrode 432 may terminate approximately half the distance from the first end to the opposite end of the elongated skin penetrating member 420 as shown in FIGS. 4B-C. Each electrode set 430A-D can further include at least one working electrode 434, wherein the working electrode 434 is positioned on the opposite end of the elongated skin penetrating member 420, and includes at least one or more contact surfaces that extends from the opposite end side of the elongated skin penetrating member 420 substantially towards the first end of the elongated skin penetrating member 420 and terminates proximate to the first end of the elongated skin penetrating member 420. The at least one working electrode 434 may include contact surfaces which terminate just before the first end of the elongated skin penetrating member 420 as shown in FIGS. 4B-C. Further, at least one counter electrode 436 can be positioned on the first end of the elongated skin penetrating member 420. The at least one counter electrode 436 can include one or more contact surfaces beginning on a first end of the elongated skin penetrating member 420 and extending towards an opposite end of the elongated skin penetrating member 420 as shown in FIG. 4B. The contact surfaces of the counter electrode 436 and the contact surfaces of the working electrode 434 may be spaced such that the electrodes 434 and 436 are electrically insulated from one another. Additionally or alternatively, the contact surfaces of the working electrode 434 and the counter electrode 436 can alternate along the elongated skin penetrating member 420 as illustrated in FIGS. 4B-C.

Analyte sensor 400 can include more than one electrode set 430, such as two, three four, five or any number of electrode sets 430. In one example implementation, at least four of electrode set 430A-D are positioned sequentially along the length of the elongated skin penetrating member 420 of an analyte sensor 400. The first of the four of electrode set 430D may be located such that the first electrode set 430D penetrates deepest into the skin of a user. A second electrode set 430C can be positioned longitudinally and/or adjacent to the first electrode set 430D of the elongated skin penetrating member 420 of the analyte sensor 400 such that the second electrode set 430C does not penetrate as far into the skin of a user as the first electrode set 430D when an analyte sensor 400 is inserted into the skin of a user. A third electrode set 430B can be positioned longitudinally and/or adjacent to, the second electrode set 430C, and along the elongated skin penetrating member 420 of the analyte sensor 400 such that the third electrode set 430B does not penetrate as far into the skin of a user as the second electrode set 430C. Additionally, a fourth electrode set 430A can be positioned longitudinally and/or adjacent to, the third electrode set 430B and along the elongated skin penetrating member 420 of the analyte sensor 400 such that the fourth electrode set 430A does not penetrate as far into the skin of a user as the third electrode set 430B.

Advantageously, implementation of at least one or more of electrode sets 430A-D of FIGS. 4A-C may be used by a disease management system such as disease management system 200 of FIG. 2, to measure and determine a gradient of a concentration of an analyte at the insertion point of the user as illustrated and described in FIG. 3B. A first electrode set 430D attached to an analyte sensor 400 can be inserted farthest into the skin of a user such that the first of electrode set 430D penetrates, for example, the adipose 308 of a user. A second electrode set 430C, third electrode set 430B, and/or fourth electrode set 430A can be sequentially inserted such that at least one of the second, third, and/or fourth of electrode sets 430A-D may be positioned at varying depths in the skin of a user, enabling the disease management system, such as disease management system 200 of FIG. 2, to measure a concentration of an analyte at varying depths in the skin and determine a gradient of an analyte concentration.

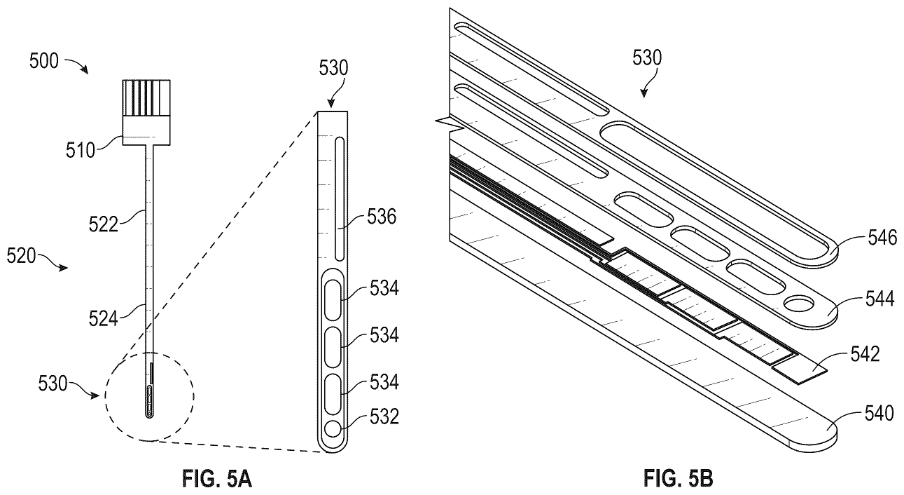

FIGS. 5A-B illustrate another example implementation of analyte sensor 500. The analyte sensor 500 can function similar to or the same as analyte sensor 120, 206, and/or 300 as illustrated in FIGS. 1-2, and 3B respectively and/or any of the analyte sensors described herein. As illustrated in FIG. 5A, the analyte sensor 500 may include a connector 510, and a skin penetrating member 520. The elongated skin penetrating member 520 can include a top portion 522 and a bottom portion 524. Additionally, analyte sensor 500 can include an electrode well 530 configured as part of the bottom portion 524 of the elongated skin penetrating member 520. Additionally, and/or alternatively, the electrode well 530 can be configured as part of the top portion 522 or any other portion of analyte sensor 500. Further, the electrode well 530 can include at least a reference electrode 532, one or more working electrodes 534, and at least one or more counter electrodes 536, and/or any combination of one or more electrodes as described herein. In an example implementation, the reference electrode 532, one or more working electrodes 534, and/or counter electrodes 536 can function the same as and/or similar to the reference electrode 432, working electrode 434, and/or counter electrodes 436 as described in FIGS. 4B-C.

As illustrated in FIG. 5A, analyte sensor 500 can include at least one reference electrode 532 positioned at the farthest portion (e.g., the tip) of the bottom portion 524 of the elongated skin penetrating member 520 such that the reference electrode 532 penetrates deepest when an analyte sensor 500 is inserted into the skin of a user. The reference electrode 532 may be positioned to penetrate deepest into the skin of a user to expose the reference electrode 532 to the highest concentration of an analyte with respect to the other electrodes of the analyte sensor 500. Further, one or more working electrodes 534 may be positioned in series (e.g. longitudinally) following the reference electrode 532 as illustrated in FIG. 5A. Additionally, and/or alternatively, the one or more working electrodes 534 may be positioned in another location along the elongated skin penetrating member 520 such as adjacent to one another at the distal end of the elongated skin penetrating member 520.

The analyte sensor 500 can include one or more of working electrode 534, for example two, three, four or more. In an example implementation, an analyte sensor 500 can include at least three of working electrode 534. The at least three of working electrode 534 may be positioned sequentially along the elongated skin penetrating member 520 such that a first of the one or more working electrodes 534 of the analyte sensor 500 penetrates deeper into the skin of a user a second working electrode 534. Additionally, a second working electrode 534 may be positioned such that the second working electrode 534 penetrates farther into the skin of a user than a third working electrode 534. Additionally and/or alternatively, the analyte sensor 500 can include more than three working electrodes 534 wherein at least one or more additional working electrodes 534 are sequentially positioned along the elongated skin penetrating member 520 of the analyte sensor 500. The analyte sensor 500 can further include at least one counter electrode 536 positioned adjacent to, and/or in sequence with, the last working electrode 534 in a sequence of electrodes such that the counter electrode 536 penetrates into the skin of a user the least of all the electrodes of the analyte sensor 500. In another implementation, the counter electrode 536, working electrode 534, and/or reference electrode 532 may be arranged in any other sequence along the analyte sensor 500.

Additionally, the electrodes may include a composite material. In an example implementation, the reference electrode 532, working electrode 534, and/or counter electrode 536 may include a composite material based on the silver/silver chloride chemistry. The reference electrode 532, working electrode 534, and/or counter electrode 536 may be created by, for example, means of screen printing, dip coating, drop casting and/or pad-printing. Further, the reference electrode 532, working electrode 534, and/or counter electrode 536 may be created using bare metal (e.g., platinum and/or gold). Additionally, the reference electrode 532, working electrode 534, and/or counter electrode 536 as illustrated in FIG. 5A can be created with the same and/or similar materials as any of the electrodes mentioned herein. to that as the modified reference electrode 532 as described herein.

Analyte sensor 500 can include several advantages with respect to, for example, at least manufacturing efficiency and/or functionality. The analyte sensor 500 may be overall easier to manufacture including with respect to electrode microfabrication and functionalization due to the implementation of an electrode well 530 defined by the one or more layers of the analyte sensor 500. The formed electrode well 530 can provide improved isolation between, for example, the working, counter, and/or reference electrodes to prevent unwanted generation of species at the electrode surfaces. Additionally, the design and manufacturing of an electrode well 530 and the placement of electrodes within the electrode well 530 of an analyte sensor 500 can reduce variations in the manufacturing volume of the measurement area for the analyte sensor 500, thereby improving accuracy of the analyte measurement. Additionally, implementation of an electrode well 530 can improve contact and adhesion between an analyte and any of the electrode surfaces, thereby reducing variations in electrical potential across the electrode surfaces.

FIG. 5B illustrates an exploded view of the electrode well 530 of an analyte sensor 500. The electrode well 530 can include, for example, a first layer 540, a second layer 542, a third layer 544, and a fourth layer 546. Additionally and/or alternatively, the electrode well 530 can include more or less layers, such as three, five, six or any other quantity of layers. The layers of analyte sensor 500 can form one or more electrode wells 530. In an example implementation, an electrode well is formed by the third layer 544 and the fourth layer 546 of the analyte sensor 500 such that the well isolates the one or more of the working electrode(s) 534 from the reference electrode 532, and/or the well isolates the one or more of working electrode(s) 534 from the one or more of counter electrode 536. In an additional implementation, the third layer 544 and fourth layer 546 form a corresponding well for at least one of the one or more electrodes 532, 534, and/or 536.

The first layer 540 of the analyte sensor 500 can be, for example, a photo-definable liquid polyimide photoresist and/or a dry film photoresist. The first layer 540 may define the substrate-geometry for the analyte sensor 500. The first layer 540 can include a varying thickness depending on the material of the first layer 540. For example, a typical thickness for the liquid polyimide photoresist can be approximately 40 microns. Additionally, and/or alternatively, the thickness for the dry film can be approximately 250 microns.

The second layer 542 may be placed on the first layer 540 of the analyte sensor 500. The second layer may include at least one or more of a working electrode, reference electrode, and counter electrode as described herein. The second layer 542 can be at least one of a list of metals including but not limited to titanium, gold and/or platinum and/or any other metal. The second layer 542 may be created, for example, by a deposited electron-beam physical vapor deposition. The process of creating the second layer 542 may result in varying thickness depending on the metals used. For example, titanium and/or gold may typically be in the range of 100-500 nm, while platinum may be approximately 150 nm.

The third layer 544 may be placed upon the second layer 542 such that the third layer defines the electrode geometries of the second layer 542. The geometries of the second layer 542 can be used to contain reagents (e.g., a silver/silver chloride composite for the reference electrode). The third layer 544 can include a photo-definable liquid polyimide photoresist and/or dry film photoresist. The thickness of the third layer 544 can be, for example, in a typical range of 9-30 nm for the liquid polyimide and/or the dry film.

The fourth layer 546 can be placed on a third layer 544 of the analyte sensor 500 such that the fourth layer defines at least one or more major wells to store an analyte limiting layer reagent. The fourth layer 546 can include a photo-definable liquid polyimide photoresist and/or dry film photoresist. The thickness of the fourth layer 546 can be, for example, in a typical range of 9-30 nm for the liquid polyimide and/or the dry film.

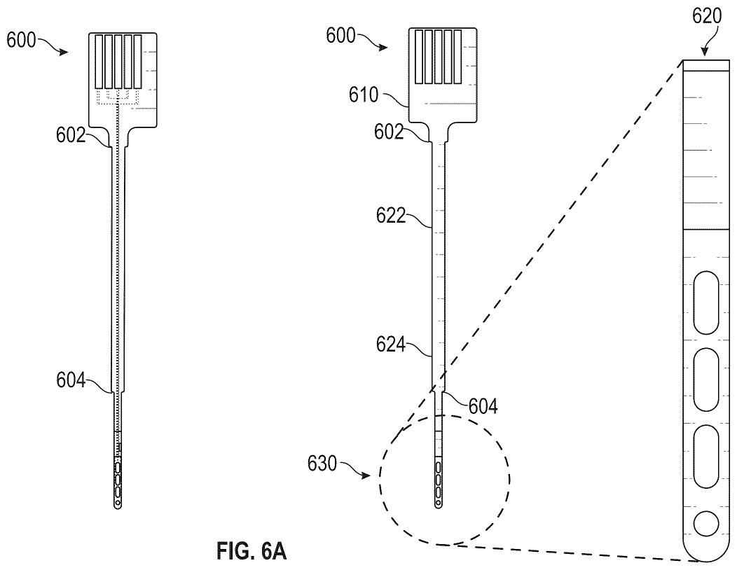

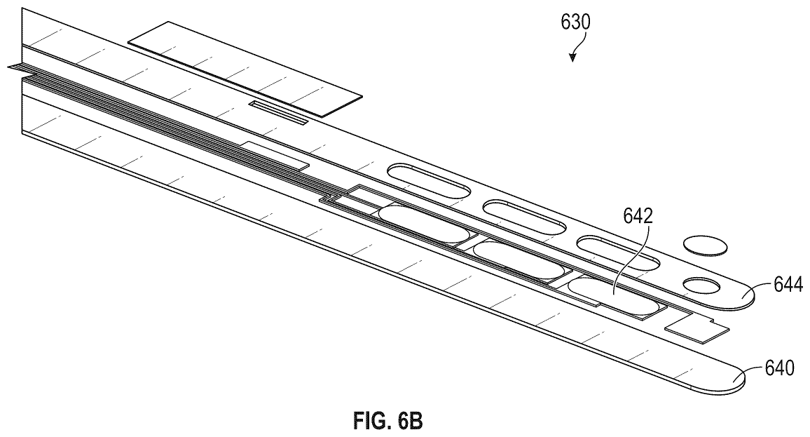

FIGS. 6A-B illustrate an example implementation of analyte sensor 600. Analyte sensor 600 can include additional variations designed to enhance the mechanical-integrity of the analyte sensor 120, 206, and/or 300 of FIGS. 1-2, and 3B respectively and/or any of the other analyte sensors described herein. The analyte sensor 600 can be functionally similar to any of the analyte sensors as described herein. Analyte sensor 600 may include some and/or any of the materials as previously described herein. The analyte sensor 600 may include, for example, a connector 610, and a skin penetrating member 620. The elongated skin penetrating member 620 can include a top portion 622 and a bottom portion 624. Further, the analyte sensor can include an electrode well 630 the same as and/or similar to the electrode well 530 as described in FIG. 5A.

As illustrated in FIG. 6A, analyte sensor 600 may include one or more narrowing portions located anywhere along the elongated skin penetrating member of the analyte sensor 600. In an example implementation, the geometry of the top portion 622 of the elongated skin penetrating member of analyte sensor 600 may include a first narrowing portion 602, approximate to the connector. Additionally, and/or alternatively, the geometry of the bottom portion 624 of the elongated skin penetrating member 620 of analyte sensor 600 may include at least a second narrowing portion 604. The addition of a first narrowing portion 602 and a second narrowing portion 604 allows for more dielectric material (photodefineable-liquid polyimide photoresist or dry film photoresist) in the connector section and near the tip of the bottom portion 624 of the elongated skin penetrating member 620. In an additional implementation, the analyte sensor 600 can include more than two narrowing portions.

Further, the analyte sensor 600 can include one or more metals in the top and/or bottom of the elongated skin penetrating member 620. In an example implementation, the bottom portion 624 can include platinum in at least some tracks (electrical leads similar to electrical leads 412 as illustrated in FIG. 4A) of the electrode well 630. When platinum is utilized in the tracks of the electrode (from the electrode well 630 to the connector 610) a tensile bending stress exists that may cause an analyte sensor 600 to bend. Advantageously, analyte sensor 600 may alleviate the tensile bending stress by depositing at least platinum on the electrode well 630 and/or including a first narrowing portion 602 and/or a second narrowing portion 604.

FIG. 6B is an exploded-view example of an electrode well 630 of analyte sensor 600. The electrode well 630 can include one or more layers. In an example implementation, the electrode well 630 of analyte sensor 600 incudes a first layer 640, a second layer 642, and a third layer 644. In some implementations, a first layer 640 and a third layer 644 can be similar to or the same as the first layer 540 and the third layer 544 respectively, as described in FIG. 5B. In some implementations, the second layer 642 can be the same as or similar to the second layer 542 described in FIG. 5B. In an example implementation, the second layer 642 can define the shapes of the electrodes for analyte sensor 600. In an example implementation, working electrodes may include an oval shape as illustrated in FIG. 6B. In an additional implementation, the second layer 642 may include any other shape for the working electrodes. Additionally, the working electrodes of the second layer 642 can include platinum within the electrode well 630.

### Example Method of Reporting an Analyte Concentration

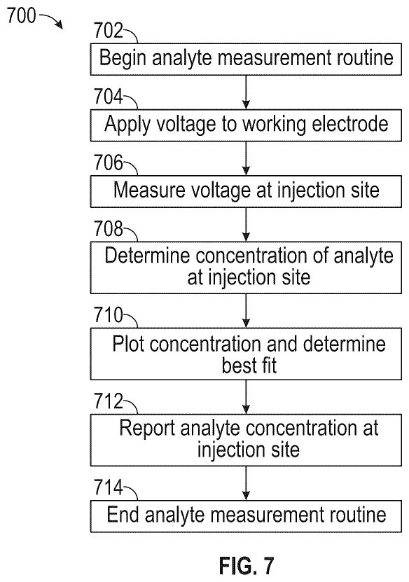

FIG. 7 illustrates an example workflow 700 for detecting and analyzing a concentration of an analyte at different depths of the skin measured from the electrodes on a skin penetrating member of an analyte sensor according to an example implementation, such as analyte sensor 120, 206, and/or 300 as illustrated in FIGS. 1-2, and 3B respectively and/or as any other analyte sensor as described herein. As an example, the controller 138 of the disease management system 100 of FIG. 1 can be configured to execute the example workflow 700 of an automated analyte measurement routine. In an example implementation, the example workflow 700 may be executed after an analyte sensor 120 penetrates the surface of the skin of a user. In an additional implementation, the example workflow 700 may be executed periodically (e.g., once per hour, once per day, once per week) after an analyte sensor 120 penetrates the surface of the skin of a user. The example workflow 700 begins at block 702.

At block 704, the controller 138 of a disease management system 100 may apply a voltage to the working electrodes of an analyte sensor 120. In an example implementation, a controller such as controller 138 of FIG. 1 applies a voltage to one working electrodes such as working electrode 432 of FIG. 4B. Additionally, and/or alternatively, the controller may apply a voltage to more than one working electrode. As described herein, the working electrode may be positioned at varying depths along an analyte sensor such that when the analyte sensor penetrates the surface of the skin of a user, the working electrodes are located at a varying depth within the user's skin with respect to one or more other working electrodes. Advantageously, having more than one working electrodes positioned at different depths within the skin of a user can be used to determine a gradient, similar to the gradient as illustrated in FIG. 3B. In another example implementation, the one or more working electrodes can be positioned on an analyte sensor such that one or more working electrodes are inserted at the same depth within the skin of a user. Advantageously, having one or more working electrodes positioned at the same depth within the skin of a user can be used to compare measurements across one or more of the working electrodes to determine an average of a concentration of an analyte at a given depth and/or to detect errors in an individual working electrode's measurement.

At block 706, the controller of a disease management system measures the voltage at the injection site of the analyte sensor. For example, the controller may measure the voltage across one or more working electrodes with respect to at least the counter electrode and/or the reference electrode of the analyte sensor such as any of the counter and/or reference electrodes as described herein. In an example implementation, the controller may measure the voltage of at least one or more working electrodes simultaneously. In an alternative implementation, the controller may measure the voltage across at least one or more working electrodes individually.

At block 708 the controller of the disease management system determines the concentration of an analyte at the injection site. The injection site can be, for example, the location where the analyte sensor penetrates the skin of a user. The controller may determine the concentration of an analyte by, for example, entering into one or more of the controller modes as described herein. For example, the controller may first enter a medium power mode before an analyte concentration calculation may be performed. The controller may determine the concentration of an analyte at the injection site by, for example, using the measured voltage for at least one working electrode. In another example implementation, the controller may determine the concentration of an analyte at varying depths of the injection site by measuring the voltage at one or more working electrodes simultaneously and/or sequentially.

At block 710 the controller may plot the concentration of an analyte and determine a best fit. The controller may receive a measured voltage from at least one or more working electrodes and calculate the concentration of an analyte. In an alternative implementation, the controller may determine a representative gradient of the concentration of analyte at the injection site for varying depths of the working electrodes of the analyte sensor.

The controller may determine a gradient of an analyte by, for example, determining the concentration of an analyte for a first working electrode, wherein the first working electrode is inserted deepest into the skin of a user, then determining the concentration of an analyte for a second working electrode, wherein the second working electrode is inserted sequentially after the first working electrode such that the second working electrode is shallower in the skin than the first working electrode, or in any other order configured by the controller.

The controller may then correlate the measured concentration of an analyte for one or more working electrodes to the respective working electrode's corresponding depth along the analyte sensor in the user's skin. Additionally, and/or alternatively, the controller may plot the concentration of an analyte as a function of depth of the analyte sensor in the user's skin. In an additional implementation, the controller may determine a gradient by averaging the calculated concentration of an analyte for a subset of at least one or more working electrodes from a plurality of working electrodes and correlating the averaged concentration of an analyte to a position of the subset of working electrodes along the analyte sensor.

At block 712 the controller may report the measured concentration of an analyte at the injection site. In an example implementation, the controller may transmit, via the one or more communication components such as communication components 140, data representing the concentration of an analyte at the injection site to, for example, other disease management systems and/or other systems as described herein. In an additional implementation, the controller may transmit data including a gradient of the measured concentration of an analyte at the injection site. After the controller transmits the measured concentration of an analyte at the injection site, the example workflow 700 for detecting and analyzing a concentration of an analyte at different depths of the skin proceeds to block 714 and ends.

### Additional Examples

Disclosed herein are additional examples of systems and methods described herein. Any of the disclosed examples may be combined in whole or in part.

Example 1: An analyte sensor to measure a concentration of at least one analyte at varying depths within a user comprising: a connector in electrical communication to a disease management system; and an elongated skin penetrating member having a length extending from a distal end and a proximal end, wherein the proximal end extends from the connector, the elongated skin penetrating member comprising: a semi-rigid substrate layer having a first side and a second side; an at least one metal layer positioned on first side of the semi-rigid substrate layer comprising a plurality of working electrodes arranged along the length of the elongated skin penetrating member, at least one reference electrode, and an at least one counter electrode, wherein the plurality of working electrodes contains at least one reference electrode therein, and the plurality of working electrodes is adjacent to the at least one counter electrode; and an at least one insulation layer surrounding the metal layer.

Example 2: The analyte sensor of Example 1, wherein the plurality of working electrodes is in electrical communication with the connector.

Example 3: The analyte sensor of Example 6, wherein a voltage is applied to the plurality of working electrodes to generate at least one measurement of the concentration of the at least one analyte.

Example 4: The analyte sensor of Example 1, wherein the second side of the semi-rigid substrate layer further comprises at least one metal layer comprising a plurality of working electrodes, at least one reference electrode, and at least one counter electrode, wherein the plurality of working electrodes contains at least one reference electrode therein, and the plurality of working electrodes is adjacent to the at least one counter electrode.

Example 5: The analyte sensor of Example 9, further comprising four working electrode sets on the distal end of the elongated skin penetrating member on the first side and four working electrode sets spanning a length of the elongated skin penetrating member on the second side.

Example 6: The analyte sensor of Example 9, wherein the plurality of working electrodes, the at least one reference electrode, and the at least one counter electrode is composed of platinum, gold, silver, silver chloride, rhodium, iridium, or a combination thereof.

Example 7: The analyte sensor of Example 1, wherein the elongated skin penetrating member is configured to be at least partially implanted in a user.

Example 8: The analyte sensor of Example 12, wherein the elongated skin penetrating member is configured to at least partially enter an adipose layer of the user.

Example 9: The analyte sensor of Example 1, wherein the elongated skin penetrating member is configured to at least partially measure a concentration of an analyte at an adipose layer of the user.

Example 10: The analyte sensor of Example 1, wherein the elongated skin penetrating member is configured to at least partially measure a concentration of an analyte at a dermis layer of the user.

Example 11: The analyte sensor of Example 1, wherein the analyte sensor is configured to be disposed in a semi-permeable electrochemical cell.

Example 12: The analyte sensor of Example 1, wherein the plurality of working electrodes contains a series of individual working electrodes electrically connected and spaced equidistantly from each other.

Example 13: The analyte sensor of Example 12, wherein the series of individual working electrodes within the plurality of working electrodes contains one reference electrode.

Example 14: The analyte sensor of Example 12, wherein the series of individual working electrodes comprises nine working electrodes.

Example 15: The analyte sensor of Example 1, wherein the plurality of working electrodes is in physical contact with the at least one counter electrode.

Example 16: An analyte measurement system for measuring a concentration of an analyte within a user, the system comprising: an analyte sensor comprising: a connector in electrical communication to a disease management system; an elongated skin penetrating member having a length extending from a distal end and an proximal end, wherein the proximal end extends from the connector, the elongated skin penetrating member comprising: a semi-rigid substrate layer having a first side and a second side; an at least one metal layer positioned on the first side of the semi-rigid substrate layer comprising a plurality of working electrodes arranged along the length of the elongated skin penetrating member, at least one reference electrode, and an at least one counter electrode, wherein the plurality of working electrodes contains at least one reference electrode therein, and the plurality of working electrodes is adjacent to the at least one counter electrode; and an at least one insulation layer surrounding the metal layer; a medication catheter configured to deliver medication to a user; a physiological sensor configured to communicate physiological values; an at least one communication components configured to transmit and receive information associated with the disease management system; and an at least one user interfacing component configured to accept and receive user input.

Example 17: A method of measuring a concentration of at least one analyte in a user at an injection site, the method comprising: inserting an elongated skin penetrating member having a plurality of working electrodes thereon into an injection site of the user to contact a bodily fluid containing at least one analyte; applying a voltage to the plurality of working electrodes wherein the plurality of working electrodes generate an electrical potential corresponding to a plurality of measurements of the at least one analyte at the injection site; communicating the plurality of measurements to a processor to determine a single analyte concentration; and reporting the single analyte concentration at the injection site to the user.

Example 18: An analyte sensor to measure a concentration of at least one analyte at varying depths within a user comprising: a connector in electrical communication to a disease management system; an elongated skin penetrating member having a length extending from a distal end and a proximal end, wherein the proximal end extends from the connector, and wherein the distal end is an electrode well, wherein the electrode well comprises: a semi-rigid substrate layer having a first side and a second side; an at least one metal layer positioned on the first side of the semi-rigid substrate layer comprising a plurality of working electrodes arranged along the length of the elongated skin penetrating member, at least one reference electrode, and an at least one counter electrode, wherein the plurality of working electrodes contains at least one reference electrode therein, and the at least one reference electrode, and the plurality of working electrodes are sequentially spaced along the electrode well; and an at least one insulation layer surrounding the metal layer.

Example 19: The analyte sensor of Example 18, wherein the plurality of working electrodes is in electrical communication with the connector.

Example 20: The analyte sensor of Example 189, wherein a voltage is applied to the plurality of working electrodes to generate at least one measurement of the concentration of the at least one analyte.

Example 21: The analyte sensor of Example 18, wherein the plurality of working electrodes, the at least one reference electrode, and the at least one counter electrode is composed of platinum, gold, silver, silver chloride, rhodium, iridium, or a combination thereof.

Example 22: The analyte sensor of Example 18, wherein the elongated skin penetrating member is configured to be at least partially implanted in a user.

Example 23: The analyte sensor of Example 21, wherein the elongated skin penetrating member is configured to at least partially enter an adipose layer of the user.

Example 24: The analyte sensor of Example 18, wherein the analyte sensor is configured to be disposed in a semi-permeable electrochemical cell.

Example 25: The analyte sensor of Example 18, wherein the plurality of working electrodes contains a series of individual working electrodes electrically connected and spaced sequentially along the electrode well.

Example 26: The analyte sensor of Example 25, wherein the series of individual working electrodes within the plurality of working electrodes contains one reference electrode.

Example 27: The analyte sensor of Example 25, wherein the series of individual working electrodes comprises three working electrodes.

Example 28: The analyte sensor of Example 18, wherein the proximal end of the elongated skin penetrating member has a first narrowing portion such that a first cross-sectional area of the proximal end of the elongated skin penetrating member nearest to the connector is greater than a second cross-sectional area of the proximal end of the elongated skin penetrating member closest to the distal end of the elongated skin penetrating member, and wherein the distal end of the elongated skin penetrating member has a second narrowing portion, such that a first cross-sectional area of the distal end of the elongated skin penetrating member nearest to the connector is greater than a second cross-sectional area of the distal end of the elongated skin penetrating member farthest from the connector.

Example 29: The analyte sensor of Example 18, wherein the plurality of working electrodes comprise of platinum.

Example 30: The analyte sensor of Example 18, wherein the reference electrode and/or the counter electrode further comprise of a composite material based on a silver/silver chloride composite.

Example 31: The analyte sensor of Example 18, wherein the counter electrode further comprises platinum or gold.

Example 32: A method of measuring a concentration of at least one analyte in a user at an injection site, the method including inserting an elongated skin penetrating member having a plurality of working electrodes arranged along the length of the elongated skin penetrating member at least into an injection site of a user, wherein the injection site includes of at least a dermis, an adipose, and at least one analyte of the user, applying a voltage to the plurality of working electrodes wherein the plurality of working electrodes generate an electrical potential corresponding to a plurality of measurements of the at least one analyte at the injection site, communicating the plurality of measurements to a processor to determine a plurality of analyte concentration values for at least the dermis and the adipose of the user, and reporting the plurality of analyte concentration values at the injection site to the user.

Example 33: The method of Example 32, wherein the plurality of working electrodes includes a series of individual working electrodes electrically connected and spaced equidistantly from each other.

Example 34: The method of Example 33, wherein the series of individual working electrodes within the plurality of working electrodes includes one reference electrode.

Example 35: The method of Example 32, wherein the series of individual working electrodes comprises nine working electrodes.

### Terminology

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. The terms “comprising,” “including,” “having,” and the like are synonymous and are used inclusively, in an open-ended fashion, and do not exclude additional elements, features, acts, operations, and so forth. That is, the above terms are to be interpreted synonymously with the phrases “having at least” or “including at least.” For example, when used in the context of a process, the term “comprising” means that the process includes at least the recited steps, but may include additional steps. When used in the context of a device, the term “comprising” means that the device includes at least the recited features or components, but may also include additional features or components. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

The term “and/or” as used herein has its broadest least limiting meaning which is the disclosure includes A alone, B alone, both A and B together, or A or B alternatively, but does not require both A and B or require one of A or one of B. As used herein, the phrase “at least one of” A, B, “and” C should be construed to mean a logical A or B or C, using a non-exclusive logical or.

The term “temperature independent” as used herein, means that the reading or measurement of the glucose level by the glucose monitoring device or the response of the glucose sensor is not affected or not substantially affected by the change of temperature. In other words, the sensor is insensitive to the change of temperature (e.g., change of body temperature as a result of physiological conditions such as hypothermia and hyperpyrexia). In some implementations, the temperature independent property of the glucose monitoring device is maintained within the operating temperature range of the device (e.g., from about 30° C. to about 45° C., from about 33° C. to about 43° C., from about 35° C. to about 41° C., or from about 36° C. to about 40° C. In some implementations, the change of temperature (per ° C.) results in less than 5%, 4%, 3%, 2%, 1%, 0.5%, 0.1% or 0.01% change in the response of the sensor, or the measurement/reading provided by the device, when all the other parameters remain the same (e.g., the glucose concentration is constant).

Conditional language used herein, such as, among others, “can,” “could,” “might,” “may,” “e.g.,” and the like, unless specifically stated otherwise, or otherwise understood within the context as used, is generally intended to convey that certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately,” “about,” “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein may include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

1. An analyte sensor to measure a concentration of at least one analyte at varying depths within a user comprising:
a connector configured to communicate with one or more hardware processors; and
an elongated skin penetrating member having a length extending from a distal end to a proximal end, wherein the proximal end extends from the connector, the elongated skin penetrating member comprising:
a semi-rigid substrate layer having a first side and a second side;
at least one metal layer positioned on first side of the semi-rigid substrate layer comprising a plurality of working electrodes arranged along the length of the elongated skin penetrating member, at least one reference electrode, and at least one counter electrode, wherein the plurality of working electrodes contains at least one reference electrode therein, and wherein the plurality of working electrodes is adjacent to the at least one counter electrode; and
at least one insulation layer surrounding the at least one metal layer.

2. The analyte sensor of claim 1, wherein the plurality of working electrodes is in electrical communication with the connector and wherein a voltage is applied to the plurality of working electrodes to generate at least one measurement of the concentration of the at least one analyte.

3. (canceled)

4. The analyte sensor of claim 1, wherein the second side of the semi-rigid substrate layer further comprises at least one metal layer comprising of the plurality of working electrodes, at least one reference electrode, and at least one counter electrode, wherein the plurality of working electrodes contains at least one reference electrode therein, and the plurality of working electrodes is adjacent to the at least one counter electrode.

5. The analyte sensor of claim 4, further comprising four working electrode sets on the distal end of the elongated skin penetrating member on the first side, and four working electrode sets spanning a length of the elongated skin penetrating member on the second side.

6. The analyte sensor of claim 1, wherein the plurality of working electrodes, the at least one reference electrode, and the at least one counter electrode comprise of at least one of platinum, gold, silver, silver chloride, rhodium, or iridium.

7. The analyte sensor of claim 1, wherein the elongated skin penetrating member is configured to be at least partially implanted in a user, and wherein the elongated skin penetrating member is further configured to measure a concentration of an analyte at an adipose layer and at a dermis layer of the user.

8.-10. (canceled)

11. The analyte sensor of claim 1, wherein the analyte sensor is configured to be disposed in a semi-permeable electrochemical cell.

12. The analyte sensor of claim 1, wherein the plurality of working electrodes contains a series of individual working electrodes electrically connected and spaced equidistantly from each other.

13. The analyte sensor of claim 12, wherein the series of individual working electrodes within the plurality of working electrodes contains one reference electrode.

14. (canceled)

15. (canceled)

16. An analyte measurement system for measuring a concentration of an analyte within a user, the system comprising:
an analyte sensor comprising:
a connector configured to communicate with one or more hardware processors;
an elongated skin penetrating member having a length extending from a distal end to a proximal end, wherein the proximal end extends from the connector, the elongated skin penetrating member comprising:
a semi-rigid substrate layer having a first side and a second side;
at least one metal layer positioned on the first side of the semi-rigid substrate layer comprising a plurality of working electrodes arranged along the length of the elongated skin penetrating member, at least one reference electrode, and an at least one counter electrode, wherein the plurality of working electrodes contains at least one reference electrode therein, and wherein the plurality of working electrodes is adjacent to the at least one counter electrode; and
at least one insulation layer surrounding the at least one metal layer;
a medication catheter configured to deliver medication to a user;
a physiological sensor configured to communicate physiological values;
an at least one communication component configured to transmit and receive information associated with the one or more hardware processors; and
an at least one user interfacing component configured to accept and receive user input.

17.-31. (canceled)

32. A method of measuring a concentration of at least one analyte in a user at an injection site, the method comprising:
inserting an elongated skin penetrating member having a plurality of working electrodes arranged along a length of the elongated skin penetrating member at least into an injection site of a user, wherein the injection site comprises of at least a dermis, an adipose, and at least one analyte of the user;
applying a voltage to the plurality of working electrodes wherein the plurality of working electrodes generate an electrical potential corresponding to a plurality of measurements of the at least one analyte at the injection site;
communicating the plurality of measurements to a processor to determine a plurality of analyte concentration values for at least the dermis and the adipose of the user; and
reporting the plurality of analyte concentration values at the injection site to the user.

33. The method of claim 32, wherein the plurality of working electrodes contains a series of individual working electrodes electrically connected and spaced equidistantly from each other.

34. The method of claim 33, wherein the series of individual working electrodes within the plurality of working electrodes contains one reference electrode.

35. (canceled)

36. The system of claim 16, wherein the plurality of working electrodes is in electrical communication with the connector and wherein a voltage is applied to the plurality of working electrodes to generate at least one measurement of the concentration of the at least one analyte.

37. The system of claim 16, wherein the second side of the semi-rigid substrate layer further comprises at least one metal layer comprising of the plurality of working electrodes, at least one reference electrode, and at least one counter electrode, wherein the plurality of working electrodes contains at least one reference electrode therein, and the plurality of working electrodes is adjacent to the at least one counter electrode.

38. The system of claim 37, wherein the analyte sensor further comprises:
four working electrode sets on the distal end of the elongated skin penetrating member on the first side; and
four working electrode sets spanning a length of the elongated skin penetrating member on the second side.

39. The system of claim 16, wherein the plurality of working electrodes, the at least one reference electrode, and the at least one counter electrode comprise of at least one of platinum, gold, silver, silver chloride, rhodium, or iridium.

40. The system of claim 16, wherein the elongated skin penetrating member is configured to be at least partially implanted in a user, and wherein the elongated skin penetrating member is further configured to measure a concentration of an analyte at an adipose layer and at a dermis layer of the user.

41. The system of claim 16, wherein the analyte sensor is configured to be disposed in a semi-permeable electrochemical cell.

42. The system of claim 16, wherein the plurality of working electrodes contains a series of individual working electrodes electrically connected and spaced equidistantly from each other.
